(19) United States
(12) Patent Application Publication
(10) Pub. No.: US 2017/0027344 A1
(43) Pub. Date: Feb. 2, 2017

(54) Head Support for Stopping Airway Disorders

(71) Applicant: Nitetronic Holding Limited, Grand Cayman (KY)

(72) Inventors: Johannes Herrnsdorf, Herdecke (DE); Hubertus Edler von Janecek, Shanghai (CN)

(21) Appl. No.: 15/223,133

(22) Filed: Jul. 29, 2016

(30) Foreign Application Priority Data
Jul. 29, 2015 (EP) 15178802.3

(51) Int. Cl. A47G 9/10; A61B 5/145; A61B 7/00; A61B 5/11; A61B 5/00; A61F 5/56; A61B 5/0205

(52) U.S. Cl. CPC A47G 9/10; A61F 5/56; A61B 5/14542; A61B 5/0205; A61B 5/11; A61B 5/4818; A61B 7/003; A47G 2009/003

(57) ABSTRACT

An airways disorder alleviating system for stopping an ongoing and/or upcoming airways disorder of a sleeping individual includes a resting surface on which the individual at least partly is to rest, a head support with an active layer configured to selectively adapt the height of the head support section-by-section, at least one spatial sensor with at least one detector in or on at least one of the resting surface and the head support for contactlessly detecting at least one of a position of a body part and a biomotion of a body part in a sensing range, and a control unit configured to actuate the active layer for selectively adapting the height of the head support depending on the at least one the detected position and the detected biomotion so that the head of the individual is moved.

### CROSS-REFERENCE TO RELATED APPLICATIONS

This application claims the priority, under 35 U.S.C. §§119, 120, 172, 363, 365, and 371, of copending European Patent App. No. 15178802.3, filed Jul. 29, 2015; the prior application is herewith incorporated by reference in its entirety.

### STATEMENT REGARDING FEDERALLY SPONSORED RESEARCH OR DEVELOPMENT

Not Applicable

### FIELD OF THE INVENTION

The present systems, apparatuses, and methods lie in the field of head supports. The present disclosure relates to pillows. Furthermore, the present systems, apparatuses, and methods relate to detecting airway disorders during sleep and measures for suppressing and/or stopping an irregular breathing event. Particularly, the disclosure relates to measures for detecting breathing activities of an individual who rests on the head support. Furthermore, the present systems, apparatuses, and methods relate to detecting a head position on a head support.

### BACKGROUND OF THE INVENTION

A head support for stopping snoring of a sleeping individual is disclosed in U.S. Patent Publication No. 2014/0310878 to Herrnsdorf, wherein the head support has a head-resting surface on which the head of a sleeping individual rests. The head support has an active layer with a configuration of neighboring deforming elements for controlling the height of the head support section-by-section. To detect a position of the head resting on the head-resting surface, a sensing layer is provided, which is between the head-resting surface and the active layer.

So far, a position of the head on the head support is detected using a sensing layer or an array of sensors that are configured to detect a change of an electrical quantity dependent upon a change of pressure applied on the head support by weight of the head. By configuring one or more sensors exposed to the weight of the head, the position of the head can be determined. The permanent impact and movement of the head on the head support may result in a continuous mechanical stress on the sensing layer, which may lead to a failure.

During sleep of an individual, pathologic effects, such as snoring and/or a sleep apnea, can reduce oxygen absorption, which may be dangerous to health of the sleeping individual. It has been found that moving the head or body of the sleeping individual during snoring or during an ongoing sleep apnea event may immediately resolve the snoring or sleep apnea event, respectively, and urges the individual to continue normal breathing.

From the prior art, various approaches are known to detect irregularities in respiration of a sleeping individual due to sleep apnea and trying to actively move the body and/or the head of the sleeping individual.

In International Patent Publication WO 2014/114438 A1, a sleeping pillow for alleviating a sleep apnea is disclosed. The sleeping pillow has an adjusting system that can be actuated for changing the position of a head resting on the sleeping pillow by way of a control device. The system has sensors for detecting the position of the head. The control device is configured so that, in addition, sensor signals from further sensors for detecting respiratory activities of a person can be detected. To detect a sleep apnea of a person using the sleeping pillow, the further sensors comprise sensors for detecting a breathing frequency or breathing movements of the chest of the person or sensors for detecting the nose dynamic pressure of a person. The adjusting systems can be actuated with respect to the sensor signals of the further sensors to alleviate or to avoid a sleep apnea event.

German Published, Non-Prosecuted Patent Application DE 10 2009 039 915 A1 discloses an apparatus for the detection and signaling of the sleep-relating breathing dysfunction of an individual. The application describes a sensor fixated at the head of an individual so that mechanical inertia at the head of the individual can be measured and analyzed to obtain head alignment information, breathing information about a breathing behavior, and snoring information about the snoring behavior of the individual.

China patent document CN 103 222 909 A discloses a pillow with air chambers. Between the surfaces, a sensor is provided that is connected to a processor. The processor is configured to collect a sleep respiratory rate of a user. The sensor is configured as a sound sensor for acquiring the number of breaths, breath frequency rate, and other sleep information from the user.

China patent document CN 102 743 246 A discloses an inflatable pillow with air chambers for changing the position of a head of an individual resting on the pillow. When a sleep apnea occurs, the individual's head is moved by locally actuating the air chambers of the pillow. Detection of the sleep apnea may be performed by a wrist assembly for detecting the patient's pulse and blood oxygen concentration or for detecting a sleeping position or by a belly assembly for detecting a swing and abdomen abdominal breathing gas flow component.

China patent document CN1557270 A discloses a pillow with inflatable air chambers that are controlled depending on an input of a respiratory sensor signal.

Korea patent document KR100711701 B1 discloses a pillow for preventing a sleep-related breathing disorder. The pillow has a number of chambers in which a pressure can be regulated. By detecting the characteristics of pressure in each of the chambers, it can be estimated whether a breathing disorder is caused by comparing the pressure of each chamber with control data indicating an optimal pressure pattern data. The pressure of the air chambers is regulated, if a breathing disorder is detected.

The approaches discussed above try to determine a sleep apnea by analyzing a sound or pressure signal or a body movement detected by external or body mounted sensors, which renders the whole system complex, unreliable, and/or inconvenient to use.

It is, therefore, an object of the present disclosure to provide an improved head support that may have robust position detection of a head of a sleeping individual. Furthermore, it is an object to provide an improved head support having a robust detection of an airway disorder during sleep of an individual. Furthermore, it is an object to reliably detect an occurrence of an airway disorder event and moving a head of a sleeping individual depending on the detection of a sleep apnea event.

Thus, a need exists to overcome the problems with the prior art systems, designs, and processes as discussed above.

### SUMMARY OF THE INVENTION

The systems, apparatuses, and methods described provide a head support for stopping airway disorders that overcomes the hereinafore-mentioned disadvantages of the heretofore-known devices and methods of this general type and that provide such features with an airways disorder alleviating system, a head support, and a method of alleviating airways disorders.

According to a first aspect, an airways disorder alleviating system for stopping an ongoing and/or upcoming airways disorder of a sleeping individual is provided, comprising a resting surface on which the individual at least partly is to rest, a head support with an active layer being configured to selectively adapt the height of the head support section by section, at least one spatial sensor with at least one detection device disposed in or on at least one of the resting surface and the head support for detecting at least one of a position of a body part and a biomotion of a body part in a sensing range in an contactless manner, and a control unit configured to actuate the active layer for selectively adapting the height of the head support depending on the at least one detected position and detected biomotion so that the head of the individual is moved.

One idea of the above airways disorder alleviating system is the use of a spatial sensor to provide a detection sensor signal by sensing, in a contactless manner, at least one of the presence and the position of a body part such as a head of the individual and a minor movement of an internal boundary and/or of an outer surface of the body part of the individual resting on the head support. A minor movement of the internal boundary and/or of the outer surface of the body part is referred herein to as a biomotion for sake of simplicity.

The spatial sensor is configured to span a detection range by establishing a field, such as at least one of an electromagnetic field, a sonic field, and/or an electrical field. A presence and/or any changes of positions of any body parts thereof within the detection range results in a distortion or any kind of measurable influence on the field that can be detected and analyzed. In case of emitted electromagnetic energy or sonic energy, electromagnetic or sonic energy is transmitted and reflected portions of the electromagnetic or sonic energy can be respectively received and detected. By analyzing reflection characteristics, the presence and/or the position of the body part and/or biomotion in or of the body part can be determined.

In case of an electrical field, the field is distorted by the presence and/or the position of a body part and/or the biomotion of the body part, which can be detected and analyzed. Particularly, capacitive effects of the impact of the presence and/or the position of a body part and/or the biomotion of the body part can be detected, measured, and/or analyzed.

The spatial sensor may be configured to detect a position of a body part of the individual, such as the head on the head support, and to actuate selected ones of deformation elements in the head support depending on the detected position of the body part so as to move the head of the individual resting on the resting surface.

The spatial sensor may be configured also to detect a biomotion, which is provided as the detection sensor signal of the spatial sensor as well as the position of a body part or an external or inner boundary on or in the body part above the resting surface.

It has been determined that a detected biomotion of a sleeping individual often has a low frequency portion, which strongly relates to its respiratory (breathing) activity. Furthermore, other portions of the detection sensor signal can be related to the heart rate, the blood pressure, and other vibrations in the individual's body caused by body functions.

Analyzing and/or filtering the detection sensor signal, therefore, relates in a more or less periodic signal, which indicates a respiratory activity, i.e., the breath-in/breath-out movements of the individual or the head position on the head support.

The configuration of the spatial sensor in, on, or under the resting area of the sleeping individual, i.e., beneath the sleeping individual, such as in the interior of the head support, allows to keep the distance between the spatial sensor and the individual's body short and substantially constant, which may lead to a good accuracy and a very accurate contactless detection with respect to the detection sensor signals. This high accuracy is particularly beneficial for reliably detecting detection sensor signals with signal portions from which indications of an upcoming and/or ongoing airways disorder event such as a snoring or a sleep apnea can be derived alone or in combination with indications of blood-oxygen saturation (SpO₂), a heart-rate/heart-beat, body movement pattern, vibrations, sounds, including snoring.

Furthermore, the configuration of the spatial sensor in, on, or under the resting area of the sleeping individual allows the focusing of the detection range towards the head or the body of an individual resting on the head support, i.e., in an upward direction for detection of at least one of a position of the head and of a biomotion of or in the head, and/or in a direction at least partly extending along a body axis of the individual resting on the head support. A possible detection range may be directed inclined to the upward direction into a direction of the body axis of the individual.

Thereby, a detection of a biomotion of body parts of another individual nearby can be excluded from detection so that the detection of the detection sensor signal is less disturbed. This allows a more reliable detection of a respiratory change stopping the breathing activities of the specific individual resting on the head support. In contrast to other techniques for the detection of respiratory activity, such as analyzing of a sound signal based on a detection of a breathing sound or a contact-bound motion detection by, for example, a chest belt or the like, the above head support allows for a more accurate acquisition of a breathing activity by excluding distortion caused by other movements of non-head body parts of the individual. Furthermore, it is possible to implement the above head support without the need of external measures for the detection of the respiratory activities.

It may be provided that the at least one spatial sensor further comprises a signal extraction device for receiving a detection sensor signal from the detection device and for extracting at least one of a position sensor signal indicating the position of the body part of the sleeping individual and a biomotion sensor signal indicating periodic body characteristics of the sleeping individual from the at least one detection sensor signal.

The control unit may be configured to detect an upcoming and/or ongoing airways disorder, particularly, a sleep apnea event, dependent upon the at least one biomotion sensor signal indicating as a body characteristics a respiratory activity of the sleeping individual.

Additionally or alternatively, the body characteristics may include at least one of a blood-oxygen saturation, a heart-rate, a body movement pattern, vibrations, and a sound emitted by the individual, wherein the control unit is configured to determine an upcoming and/or ongoing airways disorder, such as a snoring or sleep apnea event if at least one of the body characteristics is paused for a predetermined time and a predetermined body characteristics pattern is detected.

Furthermore, the detections device may be configured to have a sensing range that is directed one of upward perpendicular to the resting surface and upward inclined in a plane defined by the direction perpendicular to the resting surface and by a longitudinal direction of the resting surface.

Furthermore, the control unit may be configured to select one or more of deformation elements of the active layer to be actuated depending on the position sensor signal.

According to an exemplary embodiment, the at least one detection device of the at least one spatial sensor may be configured at one of in and on the resting surface and is displaced from the head support along a longitudinal direction of the resting surface.

Moreover, the detection device of the at least one spatial sensor may be configured at least one of on and in the interior of the head support.

Furthermore, the signal extraction device of the at least one spatial sensor may be configured in one of the head support and the control unit.

It may be provided that the at least one detection device includes an antenna, wherein the at least one spatial sensor is configured to emit radio frequency energy through an antenna as the at least one detection device and to provide the at least one detection sensor signal indicating the respiratory activity using a Doppler effect.

Additionally or alternatively, the at least one detection device may include a sonic transducer, wherein the at least one spatial sensor is configured to emit sonic, particularly, ultrasonic, energy and to provide the at least one detection sensor signal, wherein the signal extraction device is configured to analyze the detection sensor signal using an acoustic Doppler effect.

Additionally or alternatively, the at least one detection device may include an electrode configuration having a number of electrodes, wherein the detection device is configured to generate an electrical field in the sensing range, wherein the at least one spatial sensor is configured to detect a distortion of the electrical field due to at least one of a position of a body part of the sleeping individual and a biomotion of a body part of the sleeping individual and to provide at least one detection sensor signal indicating at least one of the position of the body part and the respiratory activity.

Additionally or alternatively, the detection device may include a capacitive detection device, wherein the at least one detection device includes an electrode configuration having a number of electrodes that are particularly neighbored and substantially configured along a direction, wherein the signal extraction device is configured for measuring at least one capacity between at least two of the electrodes and for providing at least one detection sensor signal dependent upon the at least one measured capacity so that at least one of a position sensor signal indicating the position of the body part of the sleeping individual and a biomotion sensor signal indicating periodic body characteristics of the sleeping individual is extractable from the at least one detection sensor signal.

By detecting an interruption of the so-detected respiratory activity for a predetermined time period, such as five seconds, an ongoing sleep apnea can be determined and a head movement initiated. Furthermore, by permanently analyzing the respiratory activity and comparing it with a provided or previously stored or extracted respiratory pattern a potentially upcoming sleep apnea can be detected.

According to a further aspect, an airways disorder alleviating system for stopping an ongoing and/or upcoming airways disorder of a sleeping individual is provided, comprising a resting surface on which the individual at least partly is to rest, a head support with an active layer being configured to selectively adapt the height of the head support section by section, at least one spatial sensor with at least one detection device disposed in or on at least one of the resting surface and the head support for detecting a position of a body part in a sensing range in a contactless manner, and a control unit configured to actuate the active layer for selectively adapting the height of the head support dependent upon the detected position so that the head of the individual is moved.

According to a further aspect, an airways disorder alleviating system for stopping an ongoing and/or upcoming airways disorder of a sleeping individual is provided, comprising a resting surface on which the individual at least partly is to rest, a head support with an active layer being configured to selectively adapt the height of the head support section by section, at least one spatial sensor with at least one detection device disposed in or on at least one of the resting surface and the head support for detecting, in a contactless manner, a biomotion of a body part in a sensing range, and a control unit configured to actuate the active layer for selectively adapting the height of the head support dependent upon the detected biomotion so that the head of the individual is moved.

According to a further aspect, a head support for stopping an ongoing and/or upcoming airways disorder of a sleeping individual is provided, comprising an active layer configured to selectively adapt the height of the head support in sections, at least one detection device of at least one spatial sensor disposed on an upper surface of the head support or in the interior of the head support for contactlessly detecting at least one of a position of a body part and a biomotion of a body part in a sensing range, and a control unit configured to actuate the active layer for selectively adapting the height of the head support in sections depending on the at least one detected position and detected biomotion so that the head of the individual is moved.

It may be provided that the detection device is configured to have the sensing range directed one of upward perpendicular to the upper surface and upward inclined in a plane defined by the direction perpendicular to the upper surface and by a longitudinal direction of the resting surface.

Moreover, the at least one detection device may include one of an antenna, wherein the at least one spatial sensor is configured to emit radio frequency energy through the antenna and to provide the at least one detection sensor signal indicating the respiratory activity using a Doppler effect, and a capacitive proximity detector with an electrode configuration having a number of electrodes, wherein the detection device is configured to generate an electrical field in the sensing range, wherein the at least one spatial sensor is configured to detect an distortion of the electrical field due to at least one of a position and a biomotion of a body part of the sleeping individual and to provide the at least one detection sensor signal.

Moreover, the electrode configuration may be disposed one of below or above the deformation elements, wherein particularly the electrode configuration has two electrode layers wherein at least one of the electrode layers is formed on the deformation elements, particularly, as a coating.

According to a further aspect, a head support for stopping an ongoing and/or upcoming airways disorder of a sleeping individual is provided, comprising an active layer configured to selectively adapt the height of the head support in sections, at least one detection device of at least one spatial sensor disposed on an upper surface of the head support or in the interior of the head support for contactlessly detecting a position of a body part in a sensing range, and a control unit configured to actuate the active layer for selectively adapting the height of the head support in sections depending on the detected position so that the head of the individual is moved.

According to a further aspect, a head support for stopping an ongoing and/or upcoming airways disorder of a sleeping individual is provided, comprising an active layer configured to selectively adapt the height of the head support in sections, at least one detection device of at least one spatial sensor disposed on an upper surface of the head support or in the interior of the head support for detecting a biomotion of a body part in a sensing range in a contactless manner, and a control unit configured to actuate the active layer for selectively adapting the height of the head support in sections depending on the detected biomotion so that the head of the individual is moved.

According to a further aspect, a method for stopping an ongoing and/or upcoming airways disorder of a sleeping individual is provided, comprising the steps of contactlessly detecting at least one of a position and a biomotion in a sensing range, particularly perpendicular to a resting surface on which the head of the individual is to rest, and to provide at least one detection sensor signal and, from the detection sensor signal, extracting at least one of a position sensor signal indicating a position of a head of the sleeping individual and a biomotion sensor signal indicating a periodic body characteristics of the sleeping individual, actuating at least one selected of the deformation elements depending on the at least one position sensor signal and the detection sensor signal so that a head of the individual resting on the resting surface is moved.

Although the systems, apparatuses, and methods are illustrated and described herein as embodied in an airways disorder alleviating system, a head support, and a method of alleviating airways disorders, it is, nevertheless, not intended to be limited to the details shown because various modifications and structural changes may be made therein without departing from the spirit of the invention and within the scope and range of equivalents of the claims. Additionally, well-known elements of exemplary embodiments will not be described in detail or will be omitted so as not to obscure the relevant details of the systems, apparatuses, and methods.

Additional advantages and other features characteristic of the systems, apparatuses, and methods will be set forth in the detailed description that follows and may be apparent from the detailed description or may be learned by practice of exemplary embodiments. Still other advantages of the systems, apparatuses, and methods may be realized by any of the instrumentalities, methods, or combinations particularly pointed out in the claims.

Other features that are considered as characteristic for the systems, apparatuses, and methods are set forth in the appended claims. As required, detailed embodiments of the systems, apparatuses, and methods are disclosed herein; however, it is to be understood that the disclosed embodiments are merely exemplary of the systems, apparatuses, and methods, which can be embodied in various forms. Therefore, specific structural and functional details disclosed herein are not to be interpreted as limiting, but merely as a basis for the claims and as a representative basis for teaching one of ordinary skill in the art to variously employ the systems, apparatuses, and methods in virtually any appropriately detailed structure. Further, the terms and phrases used herein are not intended to be limiting; but rather, to provide an understandable description of the systems, apparatuses, and methods. While the specification concludes with claims defining the systems, apparatuses, and methods of the invention that are regarded as novel, it is believed that the systems, apparatuses, and methods will be better understood from a consideration of the following description in conjunction with the drawing figures, in which like reference numerals are carried forward.

### BRIEF DESCRIPTION OF THE DRAWINGS

The accompanying figures, where like reference numerals refer to identical or functionally similar elements throughout the separate views, which are not true to scale, and which, together with the detailed description below, are incorporated in and form part of the specification, serve to illustrate further various embodiments and to explain various principles and advantages all in accordance with the systems, apparatuses, and methods. Advantages of embodiments of the systems, apparatuses, and methods will be apparent from the following detailed description of the exemplary embodiments thereof, which description should be considered in conjunction with the accompanying drawings in which:

FIG. 1 is a schematic diagram of an exemplary embodiment of a system including a head support and an underlay;

FIG. 2 is schematic diagram of the exemplary embodiment of the system of FIG. 1 with more detail of the head support and the control unit;

FIG. 3 is a cross-sectional view through the system of FIG. 1 indicating detection ranges;

FIG. 4 is an exploded, perspective view of an exemplary embodiment of a head support with an internal spatial sensor;

FIG. 5 is a schematic diagram of an exemplary embodiment of a spatial sensor;

FIG. 6 is an exploded, perspective view of a further exemplary embodiment of a head support with an internal spatial sensor; and

FIG. 7 is an exploded, perspective view of still a further exemplary embodiment of a head support with an internal spatial sensor.

### DETAILED DESCRIPTION OF THE EMBODIMENTS

As required, detailed embodiments of the systems, apparatuses, and methods are disclosed herein; however, it is to be understood that the disclosed embodiments are merely exemplary of the systems, apparatuses, and methods, which can be embodied in various forms. Therefore, specific structural and functional details disclosed herein are not to be interpreted as limiting, but merely as a basis for the claims and as a representative basis for teaching one skilled in the art to variously employ the systems, apparatuses, and methods in virtually any appropriately detailed structure. Further, the terms and phrases used herein are not intended to be limiting; but rather, to provide an understandable description of the systems, apparatuses, and methods. While the specification concludes with claims defining the features of the systems, apparatuses, and methods that are regarded as novel, it is believed that the systems, apparatuses, and methods will be better understood from a consideration of the following description in conjunction with the drawing figures, in which like reference numerals are carried forward.

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Alternate embodiments may be devised without departing from the spirit or the scope of the invention. Additionally, well-known elements of exemplary embodiments of the systems, apparatuses, and methods will not be described in detail or will be omitted so as not to obscure the relevant details of the systems, apparatuses, and methods.

Before the systems, apparatuses, and methods are disclosed and described, it is to be understood that the terminology used herein is for the purpose of describing particular embodiments only and is not intended to be limiting. The terms “comprises,” “comprising,” or any other variation thereof are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element. The terms “including” and/or “having,” as used herein, are defined as comprising (i.e., open language). The terms “a” or “an”, as used herein, are defined as one or more than one. The term “plurality,” as used herein, is defined as two or more than two. The term “another,” as used herein, is defined as at least a second or more. The description may use the terms “embodiment” or “embodiments,” which may each refer to one or more of the same or different embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact (e.g., directly coupled). However, “coupled” may also mean that two or more elements are not in direct contact with each other, but yet still cooperate or interact with each other (e.g., indirectly coupled).

For the purposes of the description, a phrase in the form “A/B” or in the form “A and/or B” or in the form “at least one of A and B” means (A), (B), or (A and B), where A and B are variables indicating a particular object or attribute. When used, this phrase is intended to and is hereby defined as a choice of A or B or both A and B, which is similar to the phrase “and/or”. Where more than two variables are present in such a phrase, this phrase is hereby defined as including only one of the variables, any one of the variables, any combination of any of the variables, and all of the variables, for example, a phrase in the form “at least one of A, B, and C” means (A), (B), (C), (A and B), (A and C), (B and C), or (A, B and C).

Relational terms such as first and second, top and bottom, and the like may be used solely to distinguish one entity or action from another entity or action without necessarily requiring or implying any actual such relationship or order between such entities or actions. The description may use perspective-based descriptions such as up/down, back/front, top/bottom, and proximal/distal. Such descriptions are merely used to facilitate the discussion and are not intended to restrict the application of disclosed embodiments. Various operations may be described as multiple discrete operations in turn, in a manner that may be helpful in understanding embodiments; however, the order of description should not be construed to imply that these operations are order dependent.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. As used herein, the terms “substantial” and “substantially” means, when comparing various parts to one another, that the parts being compared are equal to or are so close enough in dimension that one skill in the art would consider the same. Substantial and substantially, as used herein, are not limited to a single dimension and specifically include a range of values for those parts being compared. The range of values, both above and below (e.g., “+/−” or greater/lesser or larger/smaller), includes a variance that one skilled in the art would know to be a reasonable tolerance for the parts mentioned.

Herein various embodiments of the systems, apparatuses, and methods are described. In many of the different embodiments, features are similar. Therefore, to avoid redundancy, repetitive description of these similar features may not be made in some circumstances. It shall be understood, however, that description of a first-appearing feature applies to the later described similar feature and each respective description, therefore, is to be incorporated therein without such repetition.

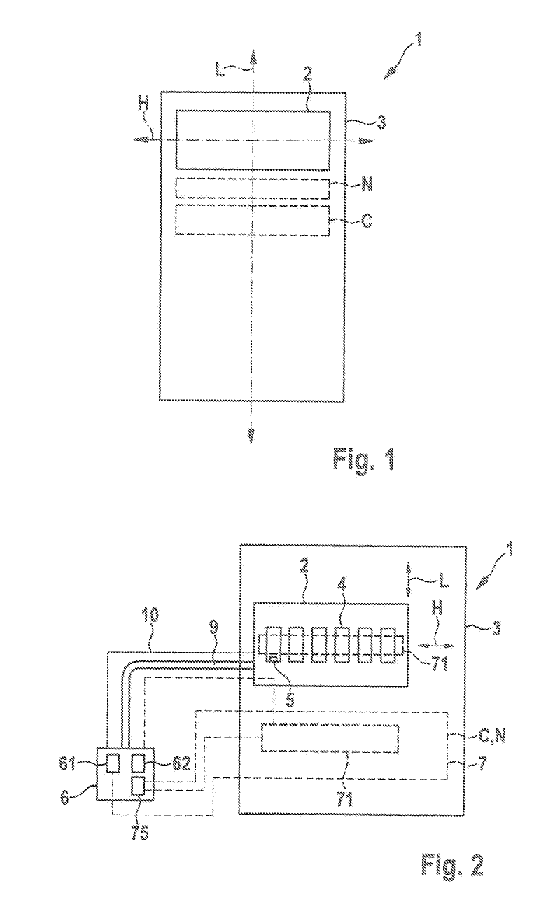

Described now are exemplary embodiments. Referring now to the figures of the drawings in detail and first, particularly to FIG. 1, there is shown a first exemplary embodiment of a system 1 including a head support 2, such as a pillow, and an underlay 3 as a resting surface, such as a mattress of a bed, which corresponds to a resting surface. The system 1 serves for an individual to rest on.

The underlay 3 has a lateral direction H and a longitudinal direction L so that the individual lying thereon with its longitudinal body axis along the longitudinal direction L of the underlay 3. The head support 2 can be freely located on the underlay 3, but it is preferred to dispose the head support 2 at or close to one end of the underlay 3 with respect to the longitudinal direction L like it is common for placing a pillow on a bed. The longitudinal L and lateral directions H of the head support 2 then substantially correspond to the longitudinal and lateral directions L, H of the underlay 3.

As shown in FIG. 2 in more detail, an airways disorder alleviating system 1 is schematically shown including the head support 2 on the underlay 3. The head support 2 can be made in the form of a pillow or the like. The head support 2 may comprise a number of deformation elements 41 such as inflatable air chambers, electrically driven height actuators, or any kind of actuators that may change its height to deform an upper surface of the head support 2 in an adaptable manner, wherein the upper surface defines a substantial plane or slightly convex surface of the head support 2. The deformation elements 41 can be disposed closely neighbored or (e.g., equally) distanced with respect to each other along the lateral direction H or as an array along a lateral and longitudinal direction H, L of the head support 2 or along any two directions different thereto. So the deformation elements 41 may form an active layer 4 capable of deforming (changing height of) the upper surface of the head support 2 in sections (i.e., section-by-section). To keep the deformation elements 41 aligned a common carrier for the deformation elements 41 can be provided, wherein the deformation elements 41 are attached to the carrier.

Each of the deformation elements 41 is configured to adapt its height with respect to a direction perpendicular to the longitudinal and lateral directions L, H depending on control signals, so that the contour of the upper surface of the head support 2 can be adapted in sections (section by section).

In case the deformation elements 41 are formed as air chambers, each air chamber can be associated with a valve 5 (exemplarily shown for just one deformation element, but provided in each deformation element 41) to inflate or deflate. Each of the air chambers may be connected with an air pump 61 (or pressured air reservoir), preferably located in a control unit 6. The control unit 6 can be located externally of the head support 2 or in the interior thereof. An air tube 9 connects to each of the air chambers 3 through a respective valve 5, which can be controlled by controller 62 of the control unit 6. The controller 62 is connected through at least one control line 10, which can be along the air tube 9 between the control unit 6 and the head support 2. Control signals are provided to the valves 5 of the head support 2 through the control line 10.

By inflating, i.e., filling an air chamber with pressured air or increasing the pressure in the air chamber, or deflating, i.e., removing air from the air chamber or reducing the air pressure in the air chamber, the height of the head support can be adapted sectionwise so that the surface of the head support 2 on which a head of the individual is resting, can be deformed so that the head is moved due to gravity, i.e., tilted along a longitudinal and/or lateral directions L, H.

The deformation elements 41 can be controlled dependent upon a detection of snoring, which can be detected by at least one sound sensor such as a microphone or by at least one pressure sensor at the deformation elements 41 (in case they are formed as air chambers).

For the position detection of a head of a sleeping individual on the head support 2, a position detector may be provided. The position detector may be configured as a sensing layer for detecting the position where the weight of a head lying on the head support 2 impacts the upper surface of the head support 2. Such a sensing layer needs to have a resolution to identify the deformation element 41 upon which the head of the individual is resting on the head support 2. This allows to selectively actuate the specific deformation element(s) 41 which is/are appropriate to have the head of the individual moved.

Alternatively or in addition to the snoring related control, a spatial sensor 7 is provided. In other exemplary embodiments, more than one spatial sensor 7 can be provided. The spatial sensor 7 has a signal extractor 75 and one or more detectors 71. The detector 71 may preferably be formed as a radio frequency transceiver (flat or rod antenna), a sonic transducer, or as a capacitive proximity detector.

If the detector 71 is formed as a radio frequency transceiver a single antenna, multiple antennas or an antenna array may be provided.

In a case where the detector 71 is formed as a capacitive proximity detector, an electrode configuration for establishing an electrical (near-)field is provided. By measuring a capacity of the electrodes, which depends on distances between the electrode configuration and a head or body part or an outer surface or inner boundary thereof, a position of the head or other body part or a biomotion can be derived. The capacitive proximity detector uses a capacitive sensing method to detect the change of capacitances with respect to the electrodes of the spatial sensor 7 due to the presence of a head or body part of an individual in a proximity thereof. This is based on that any conductive objects or object with high permittivity such as human skin, which is located nearby the capacitive proximity detector, can impact the electrode capacitance.

The detector 71 can be internal of the head support 2 to be under the head of the individual. Optionally, the detector 71 may be external from the head support 2 in or on the resting surface laterally displaced from the head support 2 along the longitudinal direction L to be under a neck or a chest (see FIG. 1) of the individual resting on the underlay 3. A neck portion N and a chest portion C are displaced from the position of the head support 2 along the longitudinal direction and indicate the positions above which the individual's neck and head, respectively, are located when resting on the underlay 3 with its head on the head support 2.

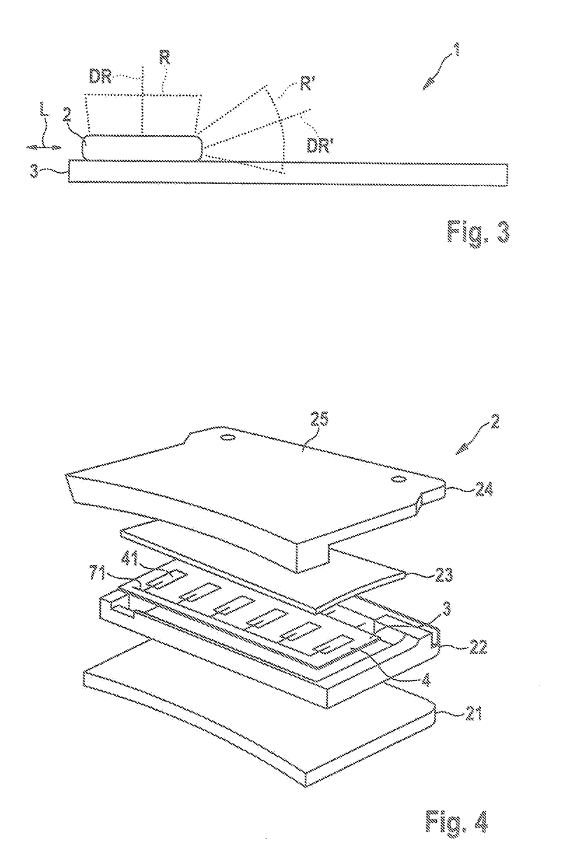

As can be seen in a cross-sectional view of FIG. 3, the detector 71 of the spatial sensor 7 has a sensing range R whose main sensing direction DR may be substantially perpendicular both to the lateral L and to the longitudinal direction H of the head support 2 or underlay 3, respectively, i.e., into a (upward) direction to the upper surface of the head support 2 or in the case of an external detector 71 into a direction to an upper surface of the underlay 3.

Additionally or alternatively, if the detector 71 of the spatial sensor 7 is disposed in the head support 2, a further main sensing direction DR′ of a further sensing range R′ may be substantially directed from the head support 2 at least partly into a longitudinal direction L towards the chest of the individual resting on the underlay 3. Particularly, the further main sensing direction DR′ can, as shown in FIG. 3, be directed inclined between the longitudinal direction L and the (upward) direction perpendicular to the lateral L and to the longitudinal direction H.

The signal extractor 75 may be located at the place of the detector 71 or remote therefrom. The signal extractor 75 is configured to extract and analyze the detection sensor signals and to provide position sensor signals and/or biomotion sensor signals.

The biomotion sensor signals are related to a biomotion of one or more external (skin) or internal boundaries in the head or body of the individual such as the motion of skin or the motion of trachea walls, vocal chords or the like. The biomotion of the one or more internal boundaries may be related to a respiratory activity of the individual so that the one or more biomotion sensor signals may have characteristics that include a relation to the respiratory action. The controller 62 of the control unit 6 is configured to receive the biomotion sensor signal from the signal extractor 75 and to detect an airways disorder, such as a sleep apnea event or a snoring, by analyzing them.

The control means 62 of the control unit 6 may be further configured to suitably compensate temperature influences on the position sensor signal and/or the biomotion sensor signal.

In case the detector 71 is incorporated in the head support 2 or has a fixed configuration with respect to the head support 2, the position sensor signals can be used to determine the position of a head on the upper surface of the head support 2. As the resolution of the above techniques applied by the detector 71 of the spatial sensor 7 is high, it provides a resolution which is appropriate to identify the one or more deformation elements 41 upon which the head of the individual is resting on the head support 2. This allows the selective driving/actuation of specific one or more of the deformation elements 41 that is/are appropriate to have the head of the individual moved in a predetermined manner.

As described above, the spatial sensor 7 may be formed as any kind of contactless sensor at least the detector 71 of which is located at (in, on, or under) the resting surface for the individual's body. The detector 71 is configured/placed to have a sensing range R that is determined by the main sensing direction DR directing from the resting surface upward so that any micro-movements in the body of the individual can be detected. In case the detector 71 is located in the head support 2, additionally, the position of the head of the sleeping individual can be detected.

Moreover, the spatial sensor 7 may be formed as any kind of contactless sensor, at least the detector 71 of which is located in the head support 2. The detector 71 is configured/placed to have a sensing range R′ that is determined by the main sensing direction DR′ directing from the head support 2 oblique in a plane defined by the longitudinal direction L and the (upward) direction perpendicular to the lateral L and to the longitudinal direction. When on the underlay 3, the main sensing direction DR′ is directed from the head support 2 inclined toward a foot end of the underlay 3.

In an exemplary embodiment, the signal extractor 75 may be disposed within the control unit 6. In this embodiment, the detector 71 is connected by wires 11 or wireless with a signal extraction device 75 in the control unit 6 either directly or through (e.g., fed-through) the head support 2 as illustrated by the dashed line.

In the following, exemplary embodiments for head supports 2 are described that can be used in an airways disorder alleviating system 1 as, e.g., shown in FIG. 2.

FIG. 4 shows an exploded view of a head support 2 with a detector 71 of a spatial sensor 7 as a radio frequency transceiver. In case the detector 71 is formed as a single radio frequency flat comb antenna, it may be disposed above the active layer 4 of the deformation elements 41 of the head support 2. In other exemplary embodiments, the detector 71 may be disposed under the active layer 4. The active layer 4 of the head support 2 corresponds to the measures by which the height of the head support 2 can be adapted section-by-section by actuating, e.g., by inflating or deflating, the deformation elements 41.

Substantially, the antenna(s) may have different configurations and can be formed as a rod, comb, V-, or flat antenna, configuration of antennas, antenna arrays, or the like and can be disposed on any portion of the head support 2 as long as detection of movements of the head on the head support 2 is not affected.

If the detector 71 has a sensing range R substantially directed in the upward direction, both the position of the head of the sleeping individual resting on the upper surface of the head support 2 and a biomotion of an external and inner boundary of the head, neck, or chest of the sleeping individual can be detected. Particularly, the biomotion of an external or inner boundary of the head of the sleeping individual can be detected independent of the position of the head resting on the head support 2.

The head support 2 comprises a number of layers that has a base layer 21 providing a rigid base support and the active layer 4 for positioning the deformation elements 41 and a frame 22 for keeping it in a predefined position. The detector 71 may be disposed above (as shown) or below the deforming elements 4 and substantially extends above them in at least one lateral direction thereof. On top of the detector 71 and the active layer 4, a buffer layer 23 for (damping) flattening the surface contour and a cover layer 24 providing the upper surface 25 on which the head of the individual resting thereon rests.

In further exemplary embodiments as indicated in FIG. 1 (dashed lines), it is provided to dispose a head support 2 (without the detector 71) on the underlay 3, wherein the detector 71 of the spatial sensor 7 is disposed external of the head support 2 on the surface of the underlay 3 or in the underlay 3. The detector 71 of the spatial sensor 7 may be disposed on the underlay 3 directly under the head support 2 or displaced from the head support 2 along a longitudinal direction L of the underlay 3. When the detector 71 is displaced from the head support 2, it may be located beneath a neck portion or chest portion of an individual resting on the underlay 3 with its head resting on the head support 2 so that movements of internal boundaries of the individual's body can be detected.

In one exemplary embodiment, the spatial sensor 7 may include a radio frequency antenna as a detector 71 that is operated to function as a radio frequency Doppler sensor. The radio frequency antenna can be used to transmit a radio frequency energy within a frequency range of 10 MHz to 100 GHz from the radio frequency antenna upward and to receive/detect the energy of a respective reflected received signal to construct the detection sensor signal by the signal extraction device 75.

The technique for detection of a biomotion, i.e., a motion of an internal boundary in the head or body of the individual, is explained in U.S. Patent Publication No. 2014/0163343 A1 to Heneghan et al., which is incorporated herein by reference and, therefore, not described in further detail.

Furthermore, the detector 71 of the spatial sensor 7 can include an ultrasonic transducer to emit and direct an ultrasonic signal in an upward direction with respect to the resting surface of the underlay 3 and/or head support 2 and to detect reflected portions of the ultrasonic signals. The ultrasonic transducer can be located above or under the active layer 4. Due to the acoustic Doppler effect, motions of internal boundaries due to respiratory activities of the individual can be detected and a respirator characteristic (breathing characteristic) can be derived therefrom.

The ultrasonic transducer of the spatial sensor 7 can be located on a specific position, in particular, in a center position of the active layer 4, and have a fan-shaped or cone-shaped emission and detection coverage. Reflected energy of the ultrasonic signal can be received by the transducer and, by analyzing the propagation delay, motion of external and internal boundaries can be detected and extracted from the received ultrasonic signal to obtain detection sensor signals to be forwarded to the controller 62.

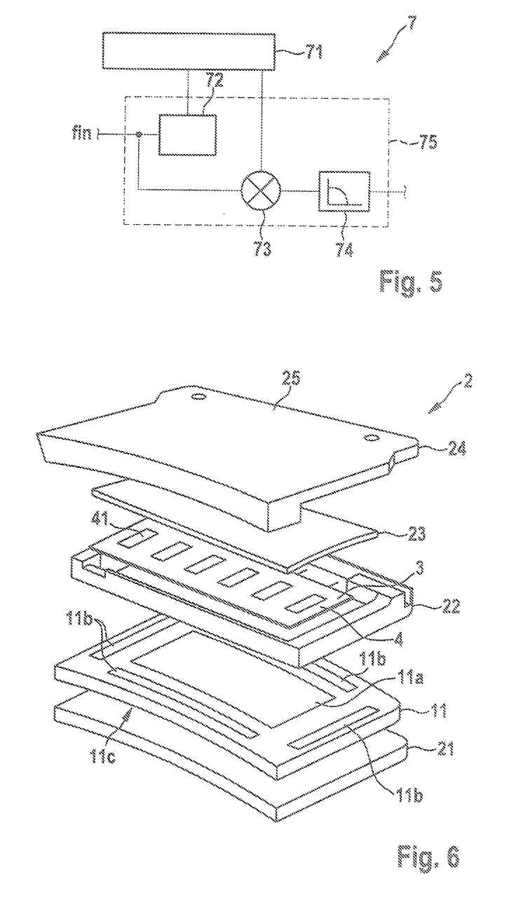

In FIG. 5, components of a radio frequency spatial sensor 7 are indicated. The components may comprise the signal extraction device 75 and be integrated together with the antenna (detector 71) into the head support 2. Alternatively the signal extraction device 75 may be a separate unit and be integrated in the control unit 6 or in the head support 2.

The radio frequency spatial sensor 7 has a transceiving antenna as the detector 71, which is connected to a radio frequency amplifier 72 that is supplied with an input radio frequency signal, which may be a pulsed or shaped radio frequency signal of a radio frequency between 10 MHz to 100 GHz. Furthermore, the antenna as well as the input radio signal are connected with a mixer 73 to detect a resulting phase shifted modulated signal indicative for the biomotion to be detected. The modulated signal is then low-pass filtered in a filter 74 to obtain the detection sensor signal to be further analyzed for a detection of a position of a head of the individual or of an airways disorder such as a snoring or a sleep apnea event.

The controller 62 is configured to drive the deformation elements 41 in case an ongoing and/or upcoming airways disorder event is detected. The deformation elements 41 are driven so that a head resting on the head support 4 will start being moved, thereby preventing the sleep apnea event to occur or causing the sleeping individual to start breathing again.

An ongoing sleep apnea event will be determined by monitoring the detection sensor signal, which has respiratory characteristics of a low frequency of 0.5 Hz to 2 Hz during normal respiration. If the detection sensor signal lacks the periodic characteristics of the low frequency for a predetermined time such as 3 to 10 seconds, then a sleep apnea event may be concluded and the actuation of the head support may be initiated.

Furthermore, the spatial sensor 7 can be configured to extract more than one detection sensor signal whose origins are located at different boundaries. The detection sensor signals can be correlated to extract the biomotion related to the respiratory activity.

Furthermore, biomotion of an internal boundary can be caused by other body functions such as heart-beat, emission of sound by vocal cords, and the like. The accord of the biomotions are detected and reflected in the detection sensor signal. By applying suitable filters 74, a heart-rate signal (signal portion in a range between 50 and 150 Hz) and a sound signal emitted by the individual (signal portion in a range above 150 Hz) can be detected.

An upcoming sleep apnea event can be determined by monitoring the detection sensor signal, which has characteristics of a low frequency of 0.5 Hz to 2 Hz during normal respiration. The low frequency portion of the detection sensor signal is compared with a predetermined respiratory pattern and, in case of a resemblance between the low frequency portion of the detection sensor signal and the predetermined respiratory pattern, an upcoming sleep apnea event may be detected.

To improve the detection of an upcoming sleep apnea event, the characteristics of other frequency portions of the detection sensor signal can be considered. In such a case, a predetermined biomotion pattern can be defined in multiple frequency ranges to be compared with respective frequency ranges to the characteristics of the detected detection sensor signal. In case of resemblance or identity, an upcoming sleep apnea event can be detected.

FIG. 6 shows an exploded view of a further head support 2 with a capacitive proximity detector as a detector 71 of a spatial sensor 7. The configuration substantially corresponds to the embodiment of FIG. 4.

The capacitive proximity detector is part of a 3D sensor technology that utilizes an electric field for proximity sensing. With an electrode configuration of electrodes, an electrical field can be generated. The electrical field may have any frequency of between 10 kHz and 1 GHz. As the wavelength is much larger than the dimensions of the electrodes, the magnetic component is practically zero and a quasi-static electrical near-field is established.

The near-field is established in a perpendicular direction above the electrode configuration. If a head of the sleeping individual intrudes the electrical field, the field becomes distorted. If the head of the sleeping individual moves in the electrical field, the distortion of the field changes. By using an electrode configuration 11 of the capacitive proximity detector, the position of the head can be clearly detected. This technique is well known in the art, e.g., from Microchip “GestIC® design guide”, 2013-2015 Microchip Technology Inc, ISBN: 978-1-63276-973-2, which is incorporated herein by reference.

The electrode configuration 11 of the capacitive proximity detector may have a plurality of electrodes forming a substantially plane array having a first layer with a rectangular inner electrode 11a and four side electrodes 11b and a second layer with a common electrode 11c. The second layer with the common electrode 11c is disposed in a plane opposite to the upper surface of the head support 2 with respect to the first layer. Each of the side electrodes 11b extends along a side of the inner electrode 11a. The extension of the common electrode 11c in the second layer substantially fully covers the inner 11a and side electrodes 11b of the first layer and is located under the first layer, i.e., opposite to the sensing range R, R′ with respect to the first layer. The common electrode 11c serves as the source of the electrical field, while the inner 11a and side electrodes 11b detect capacity changes with respect to the head positioned or moving in the electrical field. Various different configurations of electrodes 11a, 11b are possible. The electrode configuration 11 may include one or more of such electrode arrays.

The electrode configuration 11 may be located below or above the active layer 4 as long as the distance between the head and the electrode configuration 11 is within a sensing R, R′ range of the electrical field detector. Furthermore, the electrodes may be formed as a patterned conductive coating on the deformation elements 41 and/or the active layer 4 formed thereof.

The signal extraction device 75 may include a circuitry that may extract a position information of the head based on a measured indication of the distortion of the electrical field. This may, e.g., be performed by capacity measurements between the head and each of the electrodes.

Furthermore, the electrical field detector may detect periodic variations of the resulting sensor signal, which may relate to conductivity changes of the skin of the sleeping individual and or breathing activities due to slight movements of part of the skin of the head of the individual. This biomotion can be detected in a corresponding frequency range of the detection sensor signal.

For instance, the spatial sensor 7 can be configured to provide a detection sensor signal which has frequency portions related to biomotion referring to vibrations or periodic changes of external or internal boundaries of the head of the individual. These frequency portions can be related to respiratory activities and heartrate depending on their respective frequencies as described above. The detection sensor signal may be monitored correspondingly.

Furthermore, biomotion of an external or internal boundary can be caused by other body functions such as heart-beat, emission of sound by vocal cords, and the like. The accord of the biomotions are detected and reflected in the detection sensor signal. By applying suitable filters 74, a heart-rate signal (signal portion in a range between 50 and 150 Hz) and a sound signal emitted by the individual (signal portion in a range above 150 Hz) can be detected.

An upcoming sleep apnea event can be determined by monitoring the detection sensor signal, which has characteristics of a low frequency of 0.5 Hz to 2 Hz, during normal respiration. The low frequency portion of the detection sensor signal is compared with a predetermined respiratory pattern and, in case of a resemblance between the low frequency portion of the detection sensor signal and the predetermined respiratory pattern, an upcoming sleep apnea event may be detected.

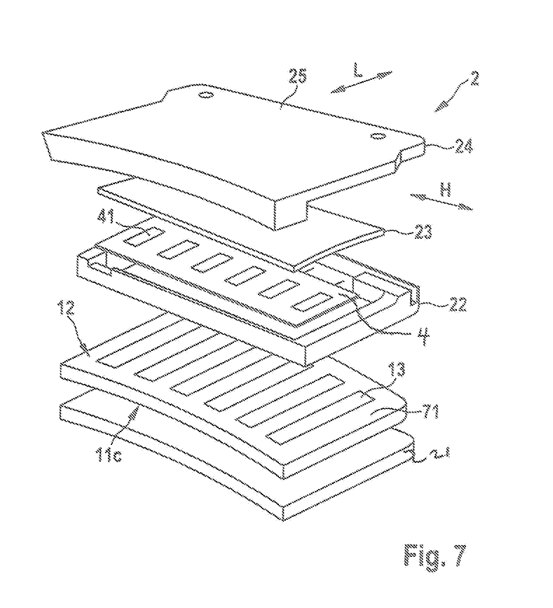

FIG. 7 shows an exploded view of a further head support 2 with a capacitive detector as the detector 71 of a spatial sensor 7.

As in the exemplary embodiments described above, the head support 2 comprises a number of layers, which includes a base layer 21 providing a rigid base support and the active layer 4 for positioning the deformation elements 41, and a frame 22 for keeping it in a predefined position. On top of the active layer 4, a buffer layer 23 may be provided for (damping) flattening the surface contour and a cover layer 24 providing the upper surface 25 on which the head of the individual resting thereon rests.

The capacitive detector 71 may have an electrode configuration 12 of a plurality of electrodes 13 forming a substantially planar array. The electrodes 13 are electrically insulated with respect to each other. The electrode configuration 12 may be formed with a number of electrodes 13 each having a same size and aligned in a row with an equal spacing distance. In other exemplary embodiments, different sizes or differing distances are appropriate as well. The electrodes 13 may have a rectangular or irregular shape and their alignment may be along the lateral direction H so that a lateral position of the head of the sleeping individual can be detected.

Detection of the position of the head or of a biomotion is performed by measuring a capacity or respectively a capacity change of one or more of the electrodes 13 and a ground/reference potential in the signal extraction device 75. Because the capacity of each of the electrodes 13 is substantially influenced by electrically conducting body parts, a detection of a position of the head or other body parts or biomotions thereof is possible.

Particularly, the proximity of a body part will result in an increase of measured capacity. Therefore, the head position is correlated to the capacitances measured at each of the electrodes 13. If the capacitance of one or more neighbored electrodes 13 increased (exceeding a predetermined threshold), the presence of the head can be assumed at the lateral position of the respective electrodes 13.

For detecting the position of a head, the electrode 13 having the highest capacitance to ground or to any other of the electrodes as a reference can be identified as the electrode above which the head is located. Detection accuracy/resolution can be improved by interpolating the measured capacitances or increasing the number of neighbored electrodes.

The electrode configuration 12 of the capacitive detector 71 may be located under (as shown) or above the active layer 4 as long as the distance between the head and the electrode configuration 12 is within a sensing R, R′ range of the electrical field detector. For instance, the electrodes may be deposited as a coating on the deforming elements 41.

Substantially, the electrodes 13 of the capacitive detector 71 may have an extension along the different positions of the head on the head support 2 to be detected and can be disposed in/on any portion of the head support 2 as long as detection of movements of the head on the head support 2 is not affected. The electrode configuration 12 of the capacitive detector 71 may substantially extend along the deforming elements 41 in at least one lateral direction thereof. Information about the lateral position of the head is important for identifying the deforming element(s) 41, which has/have to be actuated to move the head. So, the number of neighbored electrodes 13 of electrode configuration 12 is selected depending on the accuracy for position detection required for identifying the deforming elements 41 to be actuated for moving the head.

If the capacitive detector 71 is configured to have a sensing range R substantially directed in the upward direction, both the position of the head of the sleeping individual resting on the upper surface of the head support 2 and a biomotion of an external and inner boundary of the head, neck, or chest of the sleeping individual can be detected. Particularly, the biomotion of an external or inner boundary of the head of the sleeping individual can be detected independent of the position of the head resting on the head support 2.

The controller 62 is configured to drive the deformation elements 41 in case an ongoing and/or upcoming airways disorder event is detected. The deformation elements 41 are driven so that a head resting on the head support 4 will start being moved, thereby preventing the sleep apnea event to occur or causing the sleeping individual to start breathing again.

Particularly, the control unit 62 and/or the capacitive detector 71 may be configured to detect periodic variations of the resulting measured capacities, which may relate to low frequency conductivity changes of the skin of the sleeping individual due to blood flow and or slight movements of part of the skin of the head of the individual due to breathing activities. This biomotion can be detected in a corresponding frequency range of the detection sensor signal.

It is noted that various individual features of the inventive processes and systems may be described only in one exemplary embodiment herein. The particular choice for description herein with regard to a single exemplary embodiment is not to be taken as a limitation that the particular feature is only applicable to the embodiment in which it is described. All features described herein are equally applicable to, additive, or interchangeable with any or all of the other exemplary embodiments described herein and in any combination or grouping or arrangement. In particular, use of a single reference numeral herein to illustrate, define, or describe a particular feature does not mean that the feature cannot be associated or equated to another feature in another drawing figure or description. Further, where two or more reference numerals are used in the figures or in the drawings, this should not be construed as being limited to only those embodiments or features, they are equally applicable to similar features or not a reference numeral is used or another reference numeral is omitted.

The foregoing description and accompanying drawings illustrate the principles, exemplary embodiments, and modes of operation of the systems, apparatuses, and methods. However, the systems, apparatuses, and methods should not be construed as being limited to the particular embodiments discussed above. Additional variations of the embodiments discussed above will be appreciated by those skilled in the art and the above-described embodiments should be regarded as illustrative rather than restrictive. Accordingly, it should be appreciated that variations to those embodiments can be made by those skilled in the art without departing from the scope of the systems, apparatuses, and methods as defined by the following claims.

What is claimed is:

1. An airways disorder alleviating system for stopping an ongoing and/or upcoming airways disorder of a sleeping individual, the system comprising:
a resting surface on which the individual at least partly is to rest;
a head support having:
a height; and
an active layer configured to selectively adapt the height of the head support section-by-section;
at least one spatial sensor having at least one detector at least one of in and on at least one of the resting surface and the head support to contactlessly detect at least one of a position of a body part and a biomotion of a body part in a sensing range, the at least one spatial sensor being configured to use measurable field distortions of at least one of electromagnetic fields, sonic fields, and electrical fields for contactless detection; and
a control unit configured to actuate the active layer to selectively adapt the height of the head support dependent upon the at least one of the detected position and the detected biomotion so that the head of the individual is moved.

2. The system according to claim 1, wherein:
the detector creates a detection sensor signal; and
the at least one spatial sensor further comprises a signal extraction device receiving the detection sensor signal from the detector and extracting at least one of:
a position sensor signal indicating the position of the body part of the sleeping individual; and
a biomotion sensor signal indicating periodic body characteristics of the sleeping individual from the at least one detection sensor signal.

3. The system according to claim 2, wherein the detector is configured to have a sensing range directed one of upward perpendicular to the resting surface and upward inclined in a plane defined by the direction perpendicular to the resting surface and by a longitudinal direction of the resting surface.

4. The system according to claim 2, wherein the control unit is configured to detect an upcoming and/or ongoing airways disorder dependent upon the at least one biomotion sensor signal indicating a respiratory activity of the sleeping individual as a body characteristics.

5. The system according to claim 4, wherein the airways disorder is a sleep apnea event.

6. The system according to claim 4, wherein:
the body characteristics include at least one of a blood-oxygen saturation, a heart-rate, a body movement pattern, vibrations, and a sound emitted by the individual; and
the control unit is configured to determine the upcoming and/or ongoing sleep apnea event if at least one of the body characteristics is paused for a predetermined time and a predetermined body characteristics pattern is detected.

7. The system according to claim 1, wherein:
the resting surface defines a longitudinal direction; and
at least one detector of the at least one spatial sensor is one of in and on the resting surface and is displaced from the head support along the longitudinal direction of the resting surface.

8. The system according to claim 1, wherein:
the head support has an interior; and
at least one detector of the at least one spatial sensor is at least one of on and in the interior of the head support.

9. The system according to claim 2, wherein the signal extraction device of the at least one spatial sensor is disposed in one of the head support and the control unit.

10. The system according to claim 2, wherein:
the at least one detector:
creates at least one detection sensor signal;
comprises an electrode configuration having a number of electrodes;
comprises one of an antenna and a capacitive proximity detector; and
generates an electrical field in the sensing range; and
the at least one spatial sensor is configured:
to emit radio frequency energy through the antenna and to provide the at least one detection sensor signal indicating the respiratory activity; and
to detect a distortion of the electrical field due to at least one of a position and a biomotion of a body part of the sleeping individual; and
to provide at least one detection sensor signal indicating at least one of the position of the body part and the respiratory activity.

11. The system according to claim 10, wherein the at least one spatial sensor is configured to emit the radio frequency energy through the antenna and to provide the at least one detection sensor signal indicating the respiratory activity using a Doppler effect.

12. The system according to claim 1, wherein:
the detector includes a capacitive detection device;
the at least one detector includes an electrode configuration having a number of electrodes that are neighbored and disposed substantially along a given direction;
the signal extraction device is configured:
to measure at least one capacity between at least two of the electrodes; and
to provide at least one detection sensor signal dependent upon the at least one measured capacity so that at least one of a position sensor signal indicating the position of the body part of the sleeping individual and a biomotion sensor signal indicating periodic body characteristics of the sleeping individual is extractable from the at least one detection sensor signal.

13. A head support for stopping an ongoing and/or upcoming airways disorder of a sleeping individual, the head support comprising:
a support body comprising:
an interior;
an upper surface having a height; and
an active layer configured to selectively adapt the height of the upper surface in sections;
at least one spatial sensor:
comprising at least one detector at least one of on the upper surface of the head support and in the interior to contactlessly detect at least one of a position of a body part and a biomotion of a body part in a sensing range; and
configured to use measurable field distortions of at least one of electromagnetic fields, sonic fields, and electrical fields for the contactless detection; and
a control unit configured to actuate the active layer to selectively adapt the height of the head support in the sections dependent upon the at least one detected position and detected biomotion to move the head of the individual.

14. The head support according to claim 13, wherein the at least one detector is configured to have the sensing range directed one of upward perpendicular to the upper surface and upward inclined in a plane defined by the direction perpendicular to the upper surface and by a longitudinal direction of the resting surface.

15. The head support according to claim 13, wherein:
the at least one detector:
is configured to generate an electrical field in the sensing range; and
includes one of:
an antenna;
a capacitive proximity detection device comprising an electrode configuration having a number of electrodes;
the at least one spatial sensor is configured:
to emit radio frequency energy through the antenna and to provide the at least one detection sensor signal indicating the respiratory activity using a Doppler effect; and
to detect a distortion of the electrical field due to at least one of a position and a biomotion of a body part of the sleeping individual and to provide the at least one detection sensor signal.

16. The head support according to claim 13, wherein:
the at least one detector comprises:
a capacitive detection device; and
an electrode configuration having a number of electrodes neighbored and disposed substantially along a given direction; and
the signal extraction device is configured:
to measure at least one capacity between at least two of the electrodes; and
to provide at least one detection sensor signal dependent upon the at least one measured capacity so that at least one of a position sensor signal indicating the position of the body part of the sleeping individual and a biomotion sensor signal indicating periodic body characteristics of the sleeping individual is extractable from the at least one detection sensor signal.

17. The head support according to 15, wherein:
the detector is one of below and above the active layer;
the electrode configuration has two electrode layers each with one or more electrodes; and
at least one of the electrode layers is formed on the deformation elements.

18. The head support according to 17, wherein at least one of the electrode layers is formed on the deformation elements as a coated layer.

19. The head support according to 16, wherein:
the detector is one of below and above the active layer;
the electrode configuration has two electrode layers each with one or more electrodes; and
at least one of the electrode layers is formed on the deformation elements.

20. The head support according to 19, wherein at least one of the electrode layers is formed on the deformation elements as a coated layer.
